(19) United States
(12) Patent Application Publication
(10) Pub. No.: US 2006/0183764 A1
(43) Pub. Date: Aug. 17, 2006

(54) Combination of gh secret agogues and pde4 inhibitors for the treatment of alzheimers disease

(76) Inventor: Jose Luis Castro Pineiro, Bishops Stortford (GB)

Correspondence Address: MERCK AND CO., INC, P O BOX 2000, RAHWAY, NJ, 07065-0907, US

(21) Appl. No.: 10/552,367

(22) PCT Filed: Apr. 1, 2004
(86) PCT No.: PCT/GB04/01435

§ 371 Date: Oct. 3, 2005

(30) Foreign Application Priority Data
Apr. 4, 2003 (GB) 0307863.1

(51) Int. Cl. A61K 31/4747

(52) U.S. Cl. 514/278

(57) ABSTRACT

There is disclosed the treatment or prevention of diseases involving deposition of beta-amyloid in the brain, e.g. Alzheimer's disease, via the combined administration of a growth hormone secretagogue and a PDE4 inhibitor.

This invention relates to the use of methods and materials for therapeutic treatment of the human body. In particular, it provides methods of treating diseases associated with the deposition of β-amyloid in the brain, such as Alzheimer's disease, or of preventing or delaying the onset of dementia associated with such diseases.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. After secretion into the extracellular medium, the initially-soluble Aβ forms aggregates which ultimately result in the insoluble deposits and dense neuritic plaques which are the pathological characteristics of AD.

Other dementing conditions associated with deposition of Aβ in the brain include cerebral amyloid angiopathy, multi-infarct dementia, dementia pugilistica and Down syndrome.

Various interventions in the plaque-forming process have been proposed as therapeutic treatments for AD (see, for example, Hardy and Selkoe, Science, 297 (2002), 353-6), including lowering the burden of Aβ in the brain. For example, Carro et al, in Nature Medicine, 8 (2002), 1390-7, disclose that subcutaneous administration of insulin-like growth factor 1 (IGF-1) causes a reduction in the cerebral Aβ burden in certain rodents. However, some authors have questioned whether secretion of Aβ is responsible for the neuronal loss that is generally held to be the immediate cause of dementia of Alzheimer's type (see, for example, Robinson and Bishop, Neurobiology of Aging, 23 (2002), 1051-72; and also New Scientist, Feb. 1 2003, 35-37).

Growth hormone has been proposed for use in treatment of AD. Thus, U.S. Pat. No. 4,902,680 advocates the administration of growth hormone to patients in the advanced stages of AD, while WO 00/13650 discloses that increased levels of growth hormone in the brain provide a neuroprotective effect, and in particular can rescue neurons that would otherwise die as a result of an insult such as that associated with a neurodegenerative disease such as AD. The injection of growth hormone into the brain is contemplated.

Growth hormone secretagogues (GHSs) are compounds which, when administered to an animal (such as a human), stimulate or increase the release of endogenous growth hormone in the animal. Their mode of action and clinical utilities are reviewed by Ankersen et al, Drug Discovery Today, 4 (1999), 497-506; Casanueva and Dieguez, TEM, 10 (1999), 30-8; Smith et al, ibid., 10 (1999), 128-35; Betancourt and Smith, J. Anti-Aging Med., 5 (2002), 63-72; and Ghigo et al, ibid., 5 (2002), 345-56, but there is no mention of treating AD or any other neurodegenerative condition. Patents and patent applications disclosing compounds which are GHSs include U.S. Pat. No. 5,767,124, U.S. Pat. No. 5,536,716, WO 94/13696, EP 0615977B, US 5,578,593; WO 01/04119, WO 98/25897, WO 98/10653, WO 97/36873, WO 97/34604, WO 97/15574, WO 97/11697, WO 96/32943, WO 96/13265, WO 96/02530, WO 95/34311, WO 95/14666, WO 95/13069, WO 94/19367, WO 94/05634 and WO 92/16524 (all assigned to Merck & Co., Inc.); EP 1002802A, EP 0995748A, WO 98/58948, WO 98/58947 and WO 97/24369 (all assigned to Pfizer Inc.); WO 01/34593, WO 00/26252, WO 00/01726, WO 99/64456, WO 99/58501, WO 99/36431, WO 98/58950, WO 98/08492, WO 98/03473, WO 97/40071, WO 97/40023, WO 97/23508, WO 97/00894, WO 96/24587, WO 96/24580, WO 96/22997, WO 95/17423 and WO 95/17422 (all assigned to Novo Nordisk A/S); WO 96/15148 (Genentech Inc.); WO 97/22620 (Deghenghi); WO 02/32888, WO 02/32878, WO 00/49037, WO 00/10565 and WO 99/08699 (all assigned to Eli Lilly and Co.); WO 02/057241 and WO 02/056873 (both assigned to Bayer Corp.); and WO 01/85695, WO 00/54729 and WO 00/24398 (all assigned to Bristol-Myers Squibb Co.). The compounds are recommended for use in promoting the growth of food animals, and in humans for treating physiological or medical conditions characterised by a deficiency in growth hormone secretion, and medical conditions which are improved by the anabolic effects of growth hormone. In some of the above-listed disclosures, the list of treatable conditions includes AD.

The compound disclosed in the aforementioned U.S. Pat. No. 5,767,124 has been the subject of a number of clinical trials in therapeutic fields unrelated to AD (see, for example, Murphy et al, J. Bone Miner. Res., 14, (1999), 1182-8; Chapman et al, J. Clinical Endocrinology and Metabolism, 801, (1996), 4249-57; ibid., 82, (1997), 3455-63; and Svensson et al, ibid., 83, (1998), 362-9).

The 3′,5′-cyclic nucleotide phosphodiesterases (PDEs) are a class of enzymes which promote the conversion of 3′,5′-cyclic nucleotides to 5′-nucleoside monophosphates. The PDE4 isozymes are a sub-class thereof, characterised by high specificity for cAMP and sensitivity to inhibition by rolipram. Inhibition of PDE4 therefore results in raised levels of cAMP, and numerous PDE4 inhibitors are known in the art (see, for example, WO 03/108579, WO 02/060875, WO 02/074726, WO 02/098878, WO 01/46151, US 5,449,686, US 5,552,438, WO 98/45268 and WO 99/20625). The main therapeutic targets are inflammatory and/or allergic conditions such as arthritis, asthma and other pulmonary disorders, but elevation of cAMP levels also enhances cognition (see, for example, WO 02/074726 and WO 02/098878). It has been suggested that rolipram interacts with PDE4 via a high affinity rolipram binding site which is distinct from the catalytic site, or that PDE4 exists as separate isoforms having relatively high and low affinity towards rolipram, and that side-effects such as emesis found with certain PDE4 inhibitors are caused by interaction with the site or isoform having high affinity towards rolipram. In view of their anti-inflammatory and cognition-enhancing effects, PDE4 inhibitors have been proposed for use in treating Alzheimer's disease. The art thus discloses the use of PDE4 inhibitors for treating the effects of AD (e.g. neuroinflammation and cognitive impairments), but neither discloses nor suggests their utility in addressing the possible causes of AD, such as the accumulation of insoluble deposits of Aβ within the brain.

According to the invention, there is provided the use of a growth hormone secretagogue and a PDE4 inhibitor for the manufacture of a medicament for treatment or prevention of a disease associated with the deposition of β-amyloid in the brain. Said disease is typically Alzheimer's disease, cerebral amyloid angiopathy, multi-infarct, dementia pugilistica or Down syndrome, preferably Alzheimer's disease.

According to a second aspect of the invention, there is provided the use of a growth hormone secretagogue and a PDE4 inhibitor for the manufacture of a medicament for treatment, prevention or delaying the onset of dementia associated with Alzheimer's disease, cerebral amyloid angiopathy, multi-infarct dementia, dementia pugilistica or Down syndrome.

The invention also provides a method of treatment or prevention of a disease associated with the deposition of β-amyloid in the brain comprising administering to a subject in need thereof a therapeutically effective amount of a growth hormone secretagogue in combination with a therapeutically effective amount of a PDE4 inhibitor. Said disease is typically Alzheimer's disease, cerebral amyloid angiopathy, multi-infarct, dementia pugilistica or Down syndrome, preferably Alzheimer's disease.

The invention further provides a method of treating, preventing or delaying the onset of dementia associated with Alzheimer's disease, cerebral amyloid angiopathy, multi-infarct dementia, dementia pugilistica or Down syndrome comprising administering to a patient in need thereof a therapeutically effective amount of a growth hormone secretagogue in combination with a therapeutically effective amount of a PDE4 inhibitor.

As used herein, the expression “in combination with” requires that therapeutically effective amounts of both a GHS and a PDE4 inhibitor are administered to the subject, but places no restriction on the manner in which this is achieved. Thus, the two species may be combined in a single dosage form for simultaneous administration to the subject, or may be-provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred.

In a further aspect the invention provides the combination of a GHS and a PDE4 inhibitor for use in treatment or prevention of a disease associated with deposition of β-amyloid in the brain, in particular Alzheimer's disease. Said use may involve simultaneous or sequential administration of the respective therapeutic agents to a patient in need of such treatment or prophylaxis, combined in a single dosage form or in separate dosage forms.

The GHS and PDE4 inhibitor are believed to act synergistically in promoting the clearance of Aβ from the brain. It is hypothesised that the GHS causes an increase in circulating levels of growth hormone, which in turn causes increased serum levels of insulin-like growth factor 1 (IGF-1), which may promote removal of β-amyloid from the brain via transport across the blood-brain barrier. The PDE4 inhibitor is believed to further assist in this transport process.

Therefore, in a further aspect the invention provides a method for retarding, arresting or preventing the accumulation of Aβ in the brain comprising administering to a subject in need thereof a therapeutically effective amount of a growth hormone secretagogue in combination with a therapeutically effective amount of a PDE4 inhibitor. Clearance of Aβ from the brain following administration of the relevant compounds may be evidenced by altered levesl of soluble Aβ in the cerebrospinal fluid and/or the plasma. Alternatively (or additionally), imaging techniques such as magnetic resonance imaging, positron emission tomography, single photon emission computed tomography and multiphoton microscopy may be employed to monitor the extent of Aβ deposition in the brain (see, for example, Bacskai et al., J. Cereb. Blood Flow Metab., 22 (2002), 103541).

In one embodiment of the invention, the GHS and PDE4 inhibitor are administered to a patient suffering from AD, cerebral amyloid angiopathy, multi-infarct dementia, dementia pugilistica or Down syndrome, preferably AD.

In an alternative embodiment of the invention, the GHS and PDE4 inhibitor are administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8.

Within this embodiment, the GHS and PDE4 inhibitor are advantageously administered to patients who suffer impaired memory function but do not exhibit symptoms of dementia Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus. Also, Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

In a particular embodiment of the invention, GHS and PDE4 inhibitor are administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; lowered baseline hippocampal volume; elevated CSF levels of total tau; elevated CSF levels of phospho-tau; and lowered CSF levels of Aβ42.

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J Psychiatry, 141 (1984), 1356-64).

The invention further provides a kit comprising a first medicament comprising a GHS and a second medicament comprising a PDE4 inhibitor together with instructions for administering said medicaments sequentially or simultaneously to a patient suffering from AD, age-related cognitive decline, MCI, cerebral amyloid angiopathy, multi-infarct dementia, dementia pugilistica or Down syndrome.

The GHS used in the invention may be any compound which has the property of stimulating or enhancing secretion of endogenous growth hormone when administered to a subject, for example any of the compounds disclosed in the patents and patent applications listed above. However, preference is given to compounds which are suitable for oral administration.

A first class of GHSs suitable for use in the invention is that disclosed in WO 94/13696, in particular the subset thereof disclosed in EP 0615977B, the disclosure of which is incorporated herein by reference. Preferred examples of GHSs within this class include the compound of formula I: 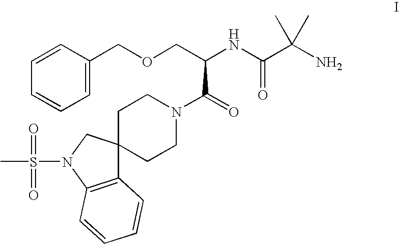
named as N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide, and pharmaceutically acceptable salts thereof in particular the methanesulfonate salt thereof, which may be prepared as described in U.S. Pat. No. 5,767,124.

A second class of GHSs suitable for use in the invention is that disclosed in U.S. Pat. No. 5,578,593, the disclosure of which is incorporated herein by reference. Preferred example of GHSs within this class include the compound of formula II: 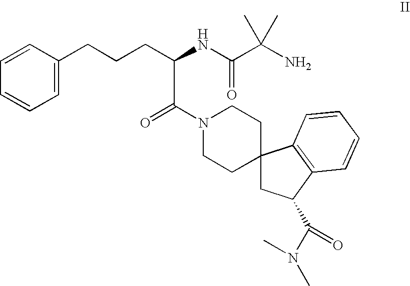
and pharmaceutically acceptable salts thereof, which may be prepared as described in U.S. Pat. No. 5,578,593.

A third class of GHSs suitable for use in the invention is that disclosed in WO 92/16524, the disclosure of which is incorporated herein by reference. Preferred example of GHSs within this class include the compounds of formulae III and IV: 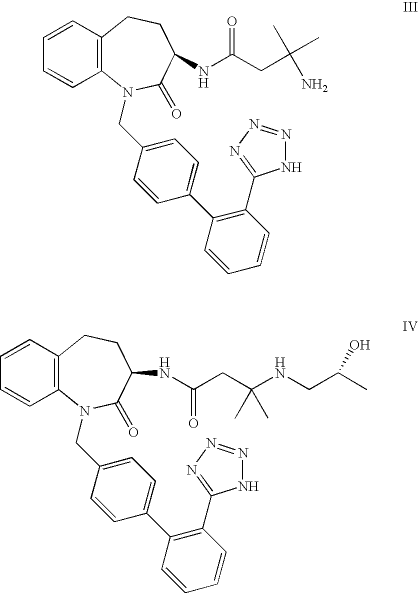
and pharmaceutically acceptable salts thereof, in particular the trifluoroacetate salts thereof, which maybe prepared as described in WO 92/16524.

A fourth class of GHSs suitable for use in the invention is that disclosed in WO 97/23508, the disclosure of which is incorporated herein by reference. Preferred examples of GHSs within this class include the compound of formula V, also known as NN703: 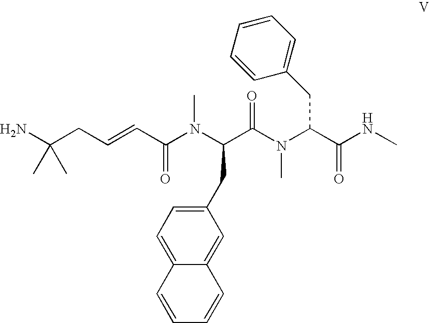
and pharmaceutically acceptable salts thereof, which may be prepared as described in WO 99/64456.

A fifth class of GHSs suitable for use in the invention is that disclosed in WO 97/24369, the disclosure of which is incorporated herein by reference. Preferred examples of GHSs within this class include the compound of formula VI: 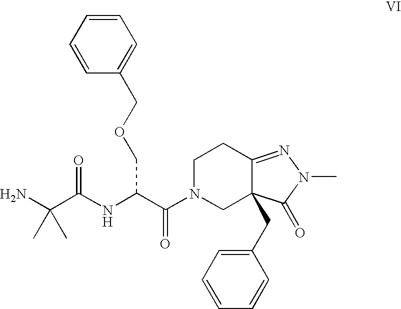
named as 2-amino-N-[2-(3a-(R)-benzyl-2-methyl-3-oxo-2,3,3a,4,6,7-hexahydro-pyrazolo[4,3-c]pyridin-5-yl)-1-(R)-benzyloxymethyl-2-oxo-ethyl]-isobutyramide, and pharmaceutically acceptable salts thereof, in particular the L-tartrate salt, also known as capromorelin, which may be prepared as described in WO 97/24369 and in Carpino et al, Bioorg. Med. Chem., 11 (2003), 581-90.

A sixth class of GHSs suitable for use in the invention is that disclosed in WO 98/58947, the disclosure of which is incorporated herein by reference. Preferred examples of GHSs within this class include the compound of formula VII: 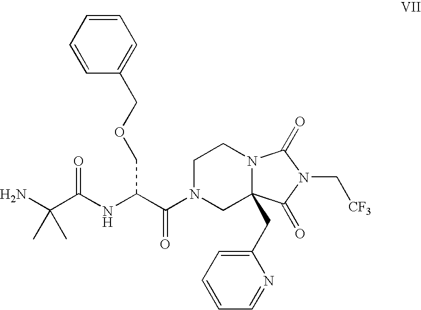
and pharmaceutically acceptable salts thereof, which may be prepared as described in WO 98/58947.

A seventh class of GHSs suitable for use in the invention is that disclosed in WO 99/08699, the disclosure of which is incorporated herein by reference. Preferred examples of GHSs within this class include the compound of formula VIII: 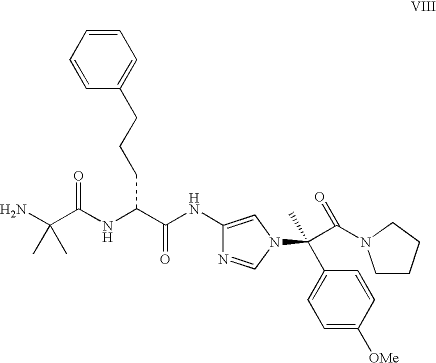
and pharmaceutically acceptable salts thereof, which may be prepared as described in WO 99/08699 and WO 02/32878.

Further GHSs suitable for use in the invention include the compound of formula IX; 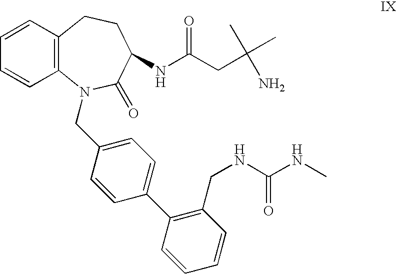
and pharmaceutically acceptable salts thereof, which may be prepared as described in De Vita et al, J. Med. Chem., 41 (1998), 1716-28, and the compound of formula X: 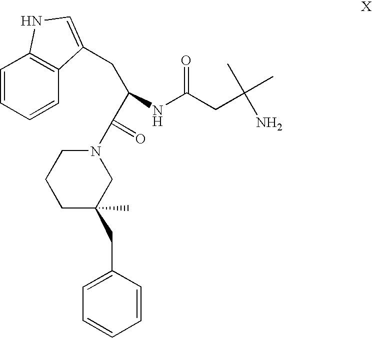
and pharmaceutically acceptable salts thereof, which may be prepared as described in Yang et al, J. Med. Chem., 41 (1998), 2439-41.

Preferably, the GHS is selected from the compounds of formulae, II, V, VI, VIII and IX depicted above, and their pharmaceutically acceptable salts. Most preferably, the GHS used in the invention is the methanesulfonate salt of the compound of formula I which is in one of the polymorphic forms described in U.S. Pat. No. 5,767,124.

The PDE4 inhibitor used in the invention may be any compound that is known to inhibit the PDE4 enzyme, or which is discovered to do so. An assay for measuring PDE4 inhibitory activity is provided in WO 01/46151. Preferably the PDE4 inhibitor does not inhibit other members of the PDE family to a significant extent at concentrations at which it inhibits PDE4 to a therapeutically-significant extent. Preferably the PDE4 inhibitor is suitable for oral administration. Preferably the PDE4 inhibitor has a relatively low affinity for the site (or isoform) which binds rolipram with high affinity, and hence (or by other means) causes minimal emesis at therapeutically effective doses. The relative affinity of a PDE4 inhibitor for the said site or isoform may be assessed as described in U.S. Pat. No. 5,998,428. It is believed that the PDE4 inhibitor can exert its beneficial action in the practice of the invention without crossing the blood-brain barrier, and so compounds having a low brain to plasma ratio, e.g. of 0.1 or 0.05 or less, or having no detectable presence in the brain, may be suitable for use in the invention.

Suitable PDE4 inhibitors include the compounds disclosed in the aforementioned WO 03/018579, WO 02/060875, WO 02/074726, WO 02/098878, WO 01/46151, U.S. Pat. No. 5,449,686, U.S. Pat. No. 5,552,438, WO 98/45268 and WO 99/20625.

A preferred PDE4 inhibitor for use in the invention is N-cyclopropyl-1-[3-(1-oxido-3-pyridinylethynyl)phenyl]-1,4-dihydro[1,8]naphthyridin4-one-3-carboxamide, or a pharmaceutically acceptable salt thereof, disclosed in WO 03/018579.

Preferred PDE4 inhibitors for use in the invention also include the class of compounds disclosed in WO 01/46151, and in particular the compound of formula XI: 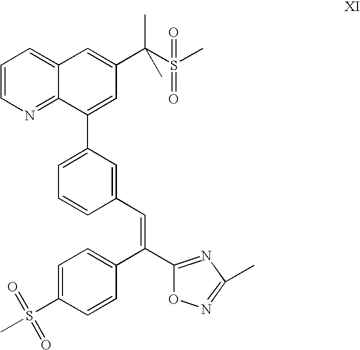
named as 6-[1-methyl-1-(methylsulfonyl)ethyl]-8-[3-[(E)-2-[3-methyl-1,2,4-oxadiazol-5-yl]-2-[4-(methylsulfonyl)phenyl]ethenyl]phenyl]quinoline, and pharmaceutically acceptable salts thereof, in particular the benzenesulfonate salt thereof; which may be prepared as described in WO 01/46151.

Preferred PDE4 inhibitors for use in the invention also include the class of compounds disclosed in U.S. Pat. No. 5,552,438, and in particular the compound of formula XII: 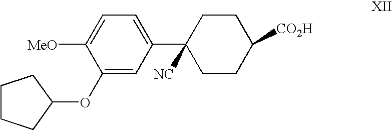
named as cis-[4-cyano4 -(3-cyclopentyloxy-4-methoxyphenyl)cyclohexane-1-carboxylic acid], and pharmaceutically acceptable salts thereof, also known as SB-207499 or cilomilast or Ariflo®, which may be prepared as described in U.S. Pat. No. 5,552,438. Further useful compounds of the same class include 2-carbomethoxy-4-cyano4(3-cyclopropylmethoxy-4difluoromethoxyphenyl)cyclohexan-1-one and cis-[4-cyano4-(3-cyclopropylmethoxy-4 -difluoromethoxyphenyl)cyclohexan-1-ol].

Further PDE4 inhibitors suitable for use in the invention include the compounds disclosed in WO 02/074726 and WO 02/098878; the compound known as MEM-1414 (Memory Pharmaceuticals Corp.); the compound R-[+]-4-[2-(3-cyclopentyloxy-4-methoxyphenyl)-2-phenylethy]pyridine, also known as CDP840 (Alexander et al, Bioorg. Med. Chem. Left., 12 (2002), 1451-6) and pharmaceutically acceptable salts thereof; the compound N-(3,5-dichloropyrid-4-yl)-3-cyclopentyloxy-4methoxybenzamide, also known as piclamilast or RP73401 (Raeburn et al, Br. J. Pharmacol., 113 (1994), 1423-31); the compound known as CP-80,633 or atizoram (Wright et al, Can. J. Physiol. Pharmacol., 75 (1997), 1001-8); the compound 1-propyl-3-(4-chlorophenyl)-xanthine, also known as LAS31025 or arofylline (see Wright et al, Br. J. Pharmacol., 126 (1999), 1863-71); the compound 3-(3-cyclopentyloxy-4-methoxybenzyl)-6-ethylamino-8-isopropyl-3H-purine hydrochloride, also known as V11294A (Gale et al, Br. J. Clin. Pharmacol., 54 (2002), 478-84); the compounds known as D4418 and D4396 (Chiroscience and Schering-Plough); the compound N-(3,5-dichloropyrid-4-yl)-[1-(4-fluorobenzyl)-5-hydroxyindole-3-yl]-glyoxylic acid amide, also known as AWD-12-281 (Baumer et al, Eur. J. Pharmacol., 446 (2002), 195-200); the compound N-(3,5-dichloropyrid-4-yl)-3-cyclopropylmethoxy-4-difluoromethoxybenzamide, also known as roflumilast (Reid, Curr. Opin. Investig. Drugs, 3 (2002), 1165-70); the 9-benzyladenine derivatives identified as NCS613, NCS675, NCS728, NCS700 and NCS706 (Raboisson et al, Eu. J. Med. Chem., 38 (2003), 199-214); the compound known as CC-3052 and other thalidomide analogues disclosed in Guckian et al, Clin. Exp. Immunol. 121 (2000), 472-9 and in Muller et al, Bioorg. Med. Chem. Lett., 8 (1998), 2669-74; and the compounds known as T440 and T2585 and analogous compounds disclosed in Uldta et al, J. Med. Chem., 42 (1999), 1088-99).

Most preferably, the PDE4 inhibitor used in the invention is the benzenesulfonate salt of the compound of formula XI or N-cyclopropyl-1-[3-(1-oxido-3-pyridinylethynyl)phenyl]-1,4-dihydro[1,8]naphthyridin-4-one-3-carboxamide, or a pharmaceutically acceptable salt thereof.

In a particularly preferred embodiment of the invention, the GHS is the methanesulfonate salt of N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide and the PDE4 inhibitor is the benzenesulfonate salt of 6-[1-methyl-1-(methylsulfonyl)ethyl]-8-[3-[(E)-2-[3-methyl-1,2,4-oxadiazol-5-yl]-2-[4-(methylsulfonyl)phenyl]ethenyl]phenyl]quinoline or N-cyclopropyl-1-[3-(1-oxido-3-pyridinylethynyl)phenyl]-1,4-dihydro[1,8]naphthyridin~one-3-carboxamide, or a pharmaceutically acceptable salt thereof.

Depending on whether they are to be administered together or separately, the GHS and PDE4 inhibitor are typically supplied as single or multiple pharmaceutical compositions comprising the active species and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and poly(ethylene glycol), and other pharmaceutical diluents, e.g. water, to form a homogeneous preformulation composition containing one or both active species, or pharmaceutically acceptable salts thereof. When referring to these preformulation compositions as homogeneous, it is meant that the active species is or are dispersed evenly throughout the composition so that the composition may be readily subdivided into equally effective unit dosage forms such as tablets, pills and capsules. This preformulation composition is then subdivided into unit dosage forms of the type described above, generally containing from 0.01 to about 500 mg of the active species. Typical unit dosage forms contain from 0.05 to 100 mg, for example 0.05, 0.1, 0.5, 1, 2, 5, 10, 25, 50 or 100 mg, of the active species. Tablets or pills of the pharmaceutical composition(s) can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) and gelatin.

Pharmaceutical compositions suitable for oral administration are preferred.

For treatment or prevention of AD, the GHS and PDE4 inhibitor may be dosed at the levels which are effective for the original purposes of the separate compounds. Thus, the GHS will typically be dosed at levels known to provide increased secretion of endogenous growth hormone in a human subject, and the PDE4 inhibitor at levels known to cause significant inhibition of the PDE4 enzyme in humans. In many cases, these dosage levels are available from the published literature, but otherwise are readily determined by standard clinical methods.

The frequency of dosing of the relevant compounds (e.g. once, twice, three times or four times per day) may be selected according to the pharmacokinetic profiles of the compounds concerned.

In the case of the preferred GHS of formula L doses of about 0.01 to 5.0 mg/kg per day, preferably about 0.05 to 2.5 mg/kg per day, more preferably about 0.1 to 1.0 mg/kg of body weight per day, may be contemplated. In particular, a dose equivalent to 5 mg, 10 mg or 25 mg of the free base may be administered orally once daily to a patient.

In the case of the PDE4 inhibitor of formula XI, doses of about 0.001 to 0.5 mg/kg per day, preferably about 0.005 to 0. I mg/kg per day, may be contemplated. In particular, doses equivalent to about 0.1 mg, 0.5 mg or 5.0 mg of the free base may be administered orally once daily to a patient.

In the case of the compound of formula XII (Ariflo®), a dose of about 5 mg, 10 mg or 15 mg per person, administered orally twice daily, may be contemplated. In the case of the compound CDP840, a dose of about 15 mg or 30 mg per person, administered orally once daily, may be contemplated. In the case of the compound V-11294A, a dose of about 300 mg per person, administered orally once daily, may be contemplated.

In a further aspect, the invention provides a pharmaceutical composition comprising, in a pharmaceutically acceptable carrier, a compound of formula I or a pharmaceutically acceptable salt thereof and a compound of formula XI or a pharmaceutically acceptable salt thereof. Preferably the compound of formula I is in the form of the methanesulfonate salt. Preferably the compound of formula XI is in the form of the benzenesulfonate salt. Preferably, the pharmaceutical composition is in a unit dose form suitable for oral administration, such as a tablet or a capsule. In a particular embodiment, said unit dose form contains the equivalent of 5, 10 or 25 mg of the free base of formula I and the equivalent of 0.1, 0.5 or 5.0 mg of the free base of formula XI.

In a further aspect, the invention provides a pharmaceutical composition comprising, in a pharmaceutically acceptable carrier, a compound of formula I or a pharmaceutically acceptable salt thereof, and the compound N-cyclopropyl-1-[3-(1-oxido-3-pyridinylethynyl)phenyl]-1,4dihydro[1,8]naphthyridin-4one-3-carboxamide, or a pharmaceutically acceptable salt thereof. Preferably the compound of formula I is in the form of the methanesulfonate salt. Preferably, the pharmaceutical composition is in a unit dose form suitable for oral administration, such as a tablet or a capsule. In a particular embodiment, said unit dose form contains the equivalent of 5, 10 or 25 mg of the free base of formula I.

1-10. (canceled)

11. A method for the treatment or prevention of a disease associated with the deposition of β-amyloid in the brain of a human patient in need thereof which comprises administering to the patient a therapeutically effective amount of a growth hormone secretagogue, or a pharmaceutically acceptable salt thereof, and a PDE4 inhibitor, or a pharmaceutically acceptable salt thereof.

12. The method of claim 11 wherein the disease is Alzheimer's disease.

13. A method for the treatment, prevention or delaying the onset of dementia associated with Alzheimer's disease, cerebral amyloid angiopathy, multi-infarct dementia, dementia pugilistica or Down syndrome in a human patient in need thereof which comprises administering to the patient a therapeutically effective amount of a growth hormone secretagogue, or a pharmaceutically acceptable salt thereof, and a PDE4 inhibitor, or a pharmaceutically acceptable salt thereof.

13. A method for the treatment, prevention or delaying the onset of mild cognitive impairment in a human patient in need thereof which comprises administering to the patient a therapeutically effective amount of a growth hormone secretagogue, or a pharmaceutically acceptable salt thereof, and a PDE4 inhibitor, or a pharmaceutically acceptable salt thereof.

15. The method of claim 14 wherein the patient possesses one or more additional risk factors for developing Alzheimer's disease selected from: a family history of the disease; a genetic predisposition to the disease; elevated serum cholesterol; adult-onset diabetes mellitus; lowered baseline hippocampal volume; elevated CSF levels of total tau; elevated CSF levels of phospho-tau; and lowered CSF levels of Aβ42.

16. The method of claim 11 wherein the growth hormone secretagogue is N-[1(R)-[(1,2-dihydro-1-methanesulfonylspiro[3H-indole-3,4′-piperidin]-1′-yl)carbonyl]-2-(phenylmethyloxy)ethyl]-2-amino-2-methyl-propanamide, or a pharmaceutically acceptable salt thereof.

17. The method of claim 16 wherein the growth hormone secretagogue is the methanesulfonate salt of N-[ 1 (R)-[(1,2-dihydro-1-methanesulfonylspiro[3H-indole-3,4′-piperidin]-1′-yl)carbonyl]-2-(phenylmethyloxy)ethyl]-2-amino-2-methyl-propanamide.

18. The method of claim 11 wherein the the PDE4 inhibitor is selected from: 6-[1-methyl-1-(methylsulfonyl)ethyl]-8-[3-[(E)-2-[3-methyl-1,2,4-oxadiazol-5-yl]-2-[4-(methylsulfonyl)phenyl]ethenyl]phenyl]quinoline; and N-cyclopropyl-1-[3-(1-oxido-3-pyridinylethynyl)phenyl]-1,4-dihydro[1,8]naphthyridin-4-one-3-carboxamide; or a pharmaceutically acceptable salt thereof.

19. The method of claim 18 wherein the the PDE4 inhibitor is the benzenesulfonate salt of 6-[1-methyl-1-(methylsulfonyl)ethyl]-8-[3-[(E)-2-[3-methyl-1,2,4-oxadiazol-5-yl]-2-[4-(methylsulfonyl)phenyl]ethenyl]phenyl]quinoline or N-cyclopropyl-1-[3-(1-oxido-3-pyridinylethynyl)phenyl]-1,4-dihydro[1,8]naphthyridin-4-one-3-carboxamide.

20. A pharmaceutical composition comprising, in a pharmaceutically acceptable carrier, a compound of formula I or a pharmaceutically acceptable salt thereof, 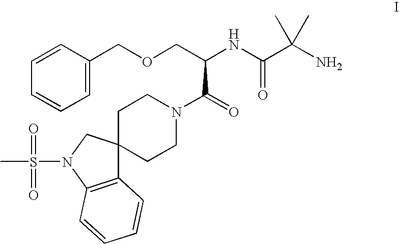
and the compound N-cyclopropyl-1-[3-(1-oxido-3-pyridinylethynyl)phenyl]-1,4-dihydro[1,8]naphthyridin-4-one-3-carboxamide, or a pharmaceutically acceptable salt thereof.
